(19) United States
(12) Patent Application Publication
(10) Pub. No.: US 2025/0236598 A1
(43) Pub. Date: Jul. 24, 2025

(54) NEW COMPOUND

(71) Applicant: SHIN-ETSU CHEMICAL CO., LTD., Tokyo (JP)

(72) Inventors: Koki KASABATA, Takasaki-shi (JP); Michihiro SUGO, Takasaki-shi (JP)

(73) Assignee: SHIN-ETSU CHEMICAL CO., LTD., Tokyo (JP)

(21) Appl. No.: 18/986,103

(22) Filed: Dec. 18, 2024

(30) Foreign Application Priority Data
Dec. 26, 2023 (JP) 2023-218787

(51) Int. Cl. C07D 251/24

(52) U.S. Cl. CPC C07D 251/24

(57) ABSTRACT

The present invention provides a compound represented by the following general formula (1). In the formula (1), V is a divalent organic group selected from any of the general formulae shown immediately below the general formula (1); “p” is 0 or 1; R¹, R², and R³ are each a hydrogen atom, a hydroxy group, an oxyalkyl group, or an alkyl group; and at least one of R¹ and R³ is a hydroxy group. This can provide a difunctional light-absorbing monomer that can be used as a monomer material for polymeric compounds.

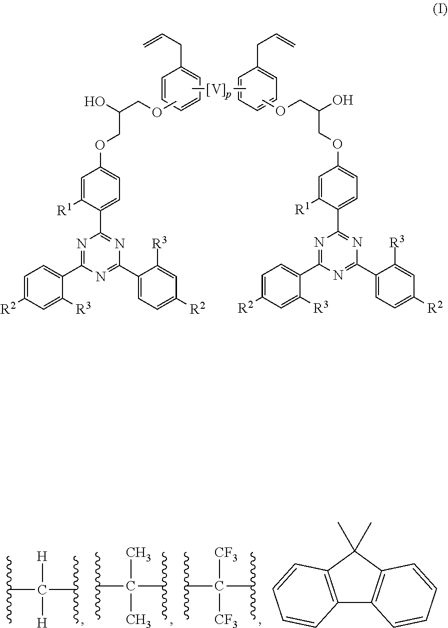

### TECHNICAL FIELD

The present invention relates to a compound having two polymerizable functional groups and two light-absorbing triazine derivatives.

### BACKGROUND ART

Light-absorbing agents have the ability to absorb light and convert it into other forms of energy such as heat, through a chemical mechanism. Utilizing this function, adding the agent can impart weather resistance to synthetic resins that deteriorate or discolor due to light. On the other hand, there are also cases where the agent is added to a synthetic resin to intentionally alter the quality of the surrounding synthetic resin by irradiating them with light and generating heat.

Examples of intentionally altering the surrounding synthetic resin by light irradiation can include a temporary adhesive material for processing semiconductors, which is peeled off by a light ray. The temporary adhesive materials for processing semiconductors are used to bond a semiconductor substrate to a support such as silicon or glass via an adhesive layer to prevent the semiconductor substrate from being damaged in processes such as grinding the back surface and forming a TSV or an electrode on the back surface. A semiconductor substrate laminated with the adhesives must be easily peelable at the final stage in semiconductor processing. In order to provide such a releasability, it is often to adopt a method to add a light-absorbing agent to the adhesive layer.

Patent Document 1 discloses temporary adhesive materials using such a temporary adhesive for semiconductors. That is, the document discloses temporary adhesive materials that are composed of an adhesive component and a light-absorbing component and can be peeled off by light irradiation.

However, as described in Patent Document 1, the light-absorbing component to be added is often added as a single substance, and there are cases where the light-absorbing component may elute and cannot maintain its original releasing function when exposed to an organic solvent during the semiconductor component manufacturing process.

### CITATION LIST

### Patent Literature

Patent Document 1: WO 2020/105586 A1

### SUMMARY OF INVENTION

### Technical Problem

The present invention has been made in view of the above-described problem. An object of the present invention is to provide a difunctional light-absorbing monomer that can be used as a monomer material for polymeric compounds.

### Solution to Problem

To solve the problem above, the present invention provides a compound represented by the following general formula (1)

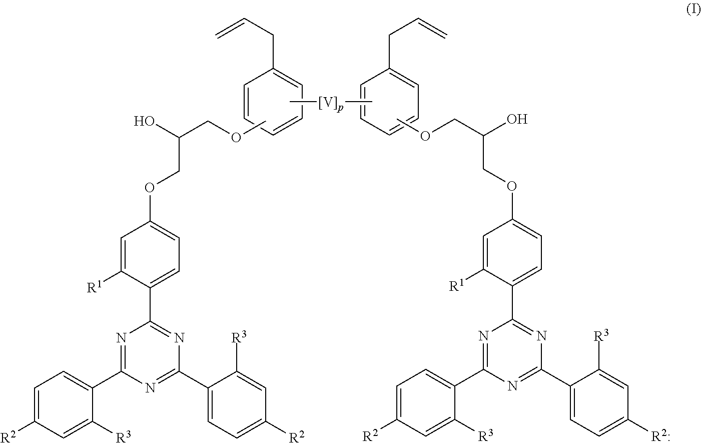

wherein, V is a divalent organic group selected from any of the following general formulae

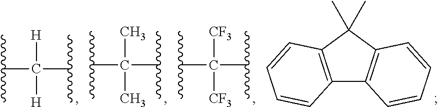

“p” is 0 or 1; R¹, R², and R³ are each a hydrogen atom, a hydroxy group, an oxyalkyl group, or an alkyl group; and at least one of R¹ and R³ is a hydroxy group.

The inventive compound can provide a difunctional light-absorbing monomer that can be used as a monomer material having two polymerizable functional groups and two light-absorbing triazine derivatives for polymer synthesis.

The inventive compound is preferably a light-absorbing compound.

When the inventive compound is a light-absorbing compound, by using it as a temporary adhesive material for processing semiconductors for example, the light-absorbing component does not elute and can maintain its releasing function even when exposed to an organic solvent.

Further, the inventive compound preferably has one or more maximum absorption wavelengths λmax of light between 200 nm and 1000 nm.

When such an inventive compound is used as a temporary adhesive material for processing semiconductors for example, a semiconductor substrate laminated with an adhesive can be easily peeled off by light irradiation in semiconductor processing.

### Advantageous Effects of Invention

As described above, the present invention can provide a compound having two polymerizable functional groups and two light-absorbing triazine derivatives. When the inventive compound is used as a temporary adhesive material for processing semiconductors, the light-absorbing component does not elute even when exposed to an organic solvent, and the releasing function can be maintained. Thereby, the semiconductor substrate laminated with the adhesive can be easily peeled off by light irradiation at the final stage in semiconductor processing.

### DESCRIPTION OF EMBODIMENTS

As described above, it has been desired to develop a compound that does not elute the light-absorbing component even when exposed to an organic solvent and can maintain its releasing function.

As a result of their diligent study of the above problem, the inventors found a compound having two polymerizable functional groups and two light-absorbing triazine derivatives, and have completed the present invention.

That is, the present invention is a compound represented by the following general formula (1)

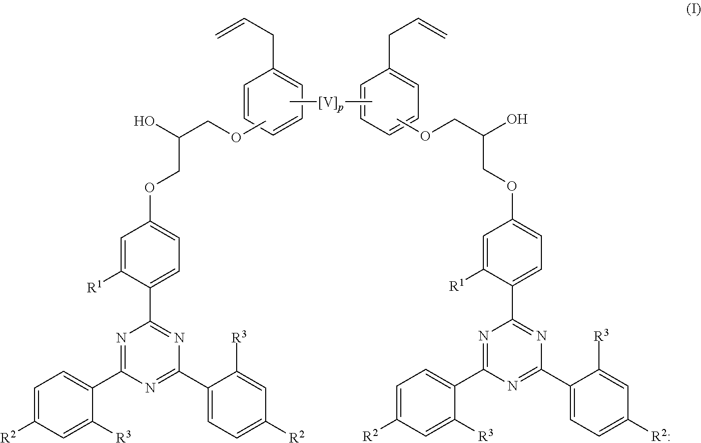

wherein, V is a divalent organic group selected from any of the following general formulae

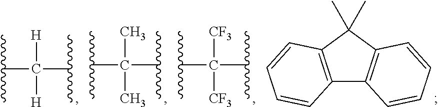

“p” is 0 or 1; R¹, R², and R³ are each a hydrogen atom, a hydroxy group, an oxyalkyl group, or an alkyl group; and at least one of R¹ and R³ is a hydroxy group.

The present invention will be described in detail below, but the present invention is not limited thereto.

The inventive compound having two polymerizable functional groups and two light-absorbing triazine derivatives is represented by the general formula (1) above. In the formula (1), V is a divalent organic group selected from any of the following formulae.

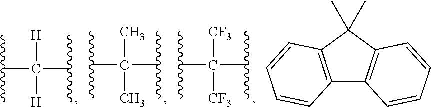

“p” is 0 or 1. Further, R¹, R², and R³ are each a hydrogen atom, a hydroxy group, an oxyalkyl group, or an alkyl group, and at least one of R¹ and R³ is a hydroxy group.

Examples of the oxyalkyl groups include hydrocarbon groups having 1 to 7 carbon atoms such as a methoxy group, an ethoxy group, a propoxy group, an isopropoxy group, a butoxy group, an isobutoxy group, a tert-butoxy group, a pentyloxy group, a neopentyloxy group, a hexyloxy group, and a heptyloxy group. Further, a typical example is a methoxy group.

Examples of the alkyl group include alkyl groups having 1-7 carbon atoms such as a methyl group, an ethyl group, a propyl group, an isopropyl group, a butyl group, an isobutyl group, a tert-butyl group, a pentyl group, a neopentyl group, a hexyl group, and a heptyl group. Further, a typical example is a methyl group.

Examples of the compound represented by such a general formula (1) include compounds represented by the following formulae (2) to (13), as typical examples.

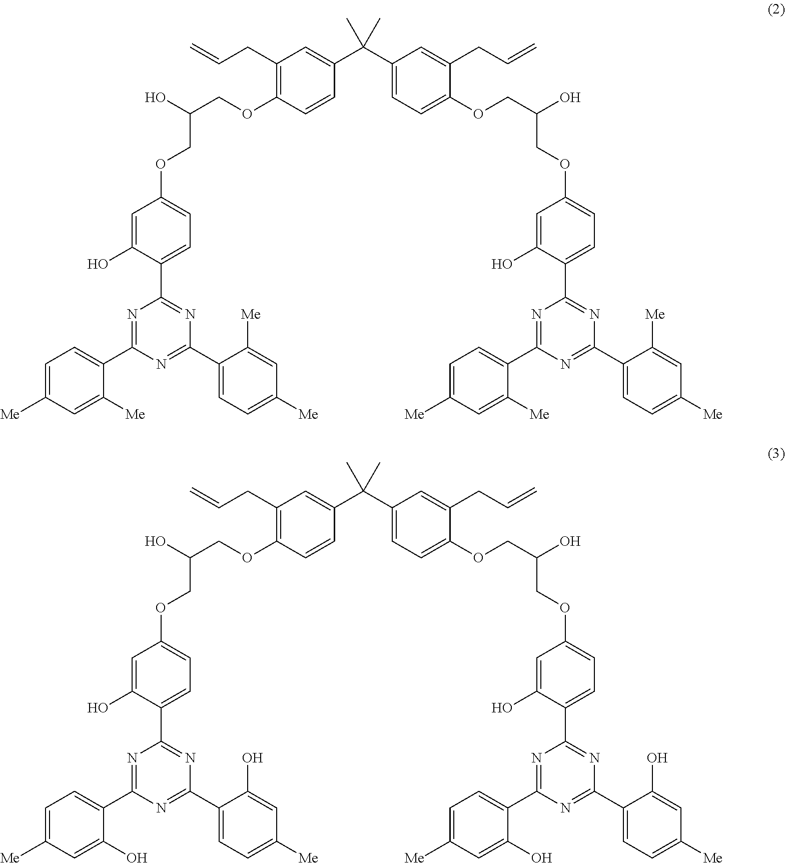

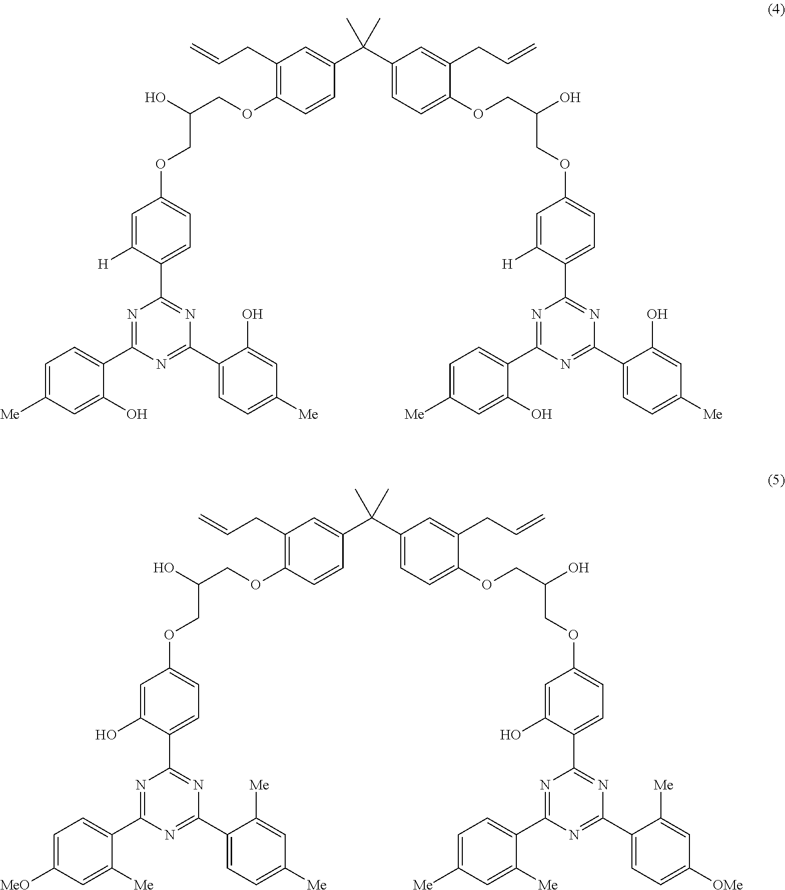

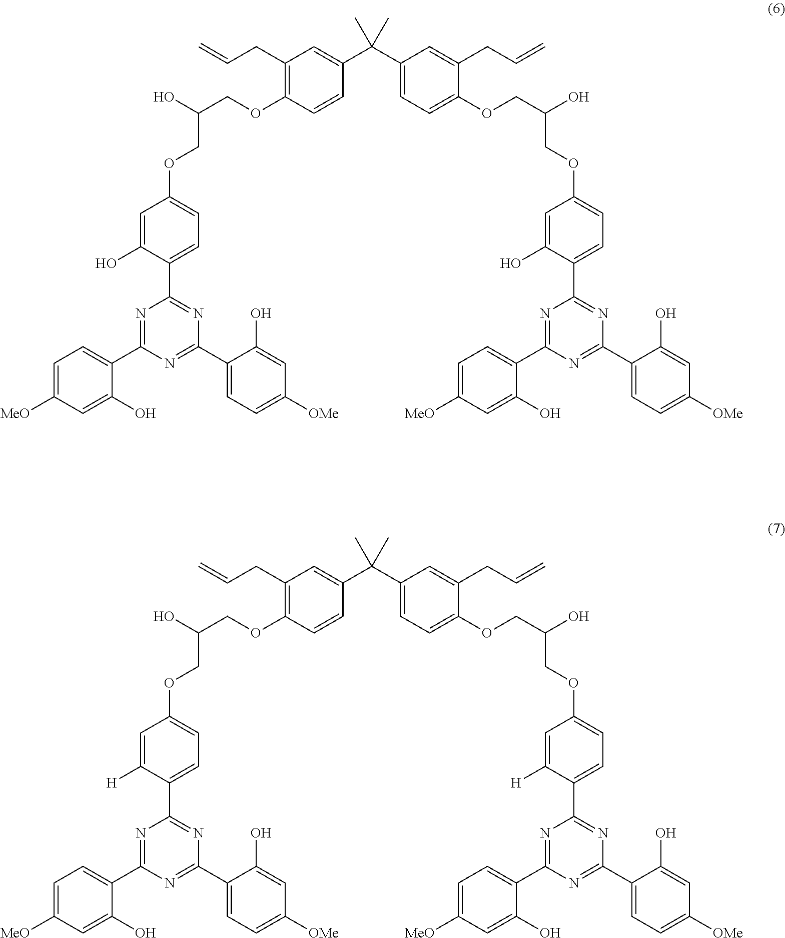

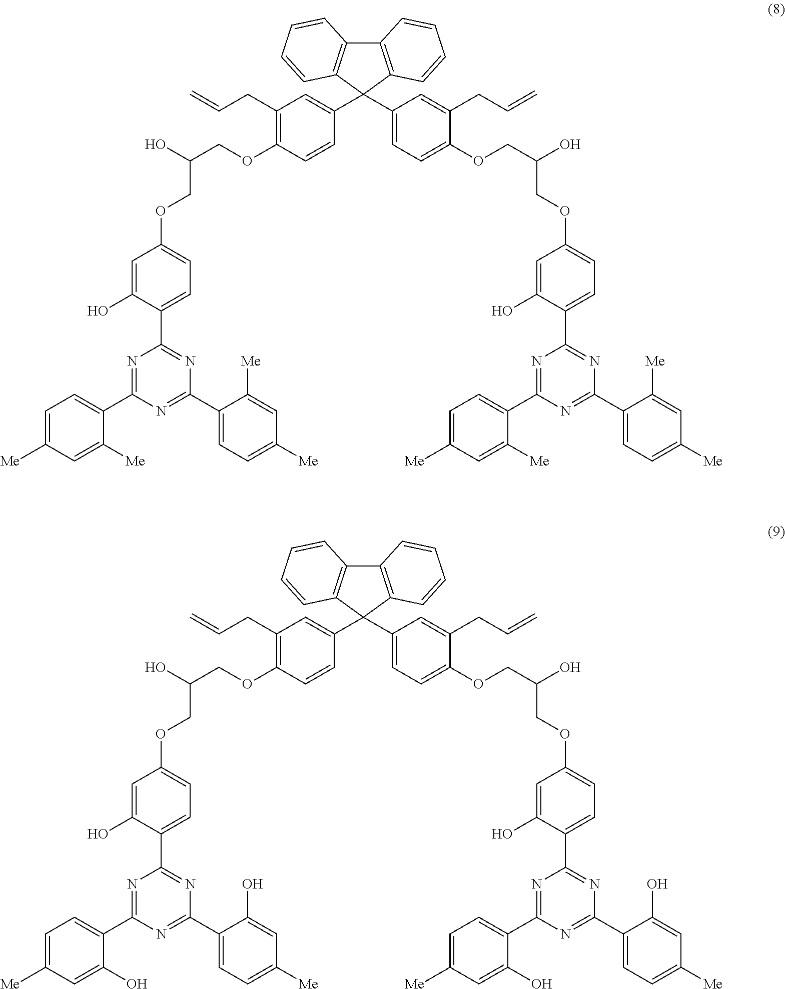

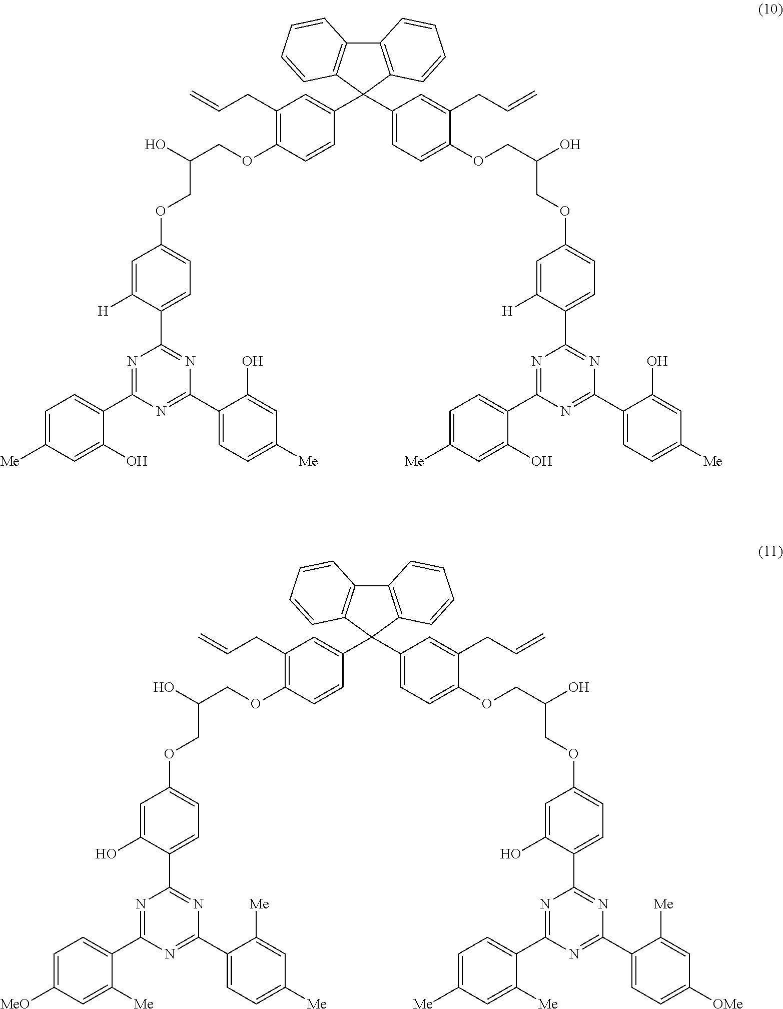

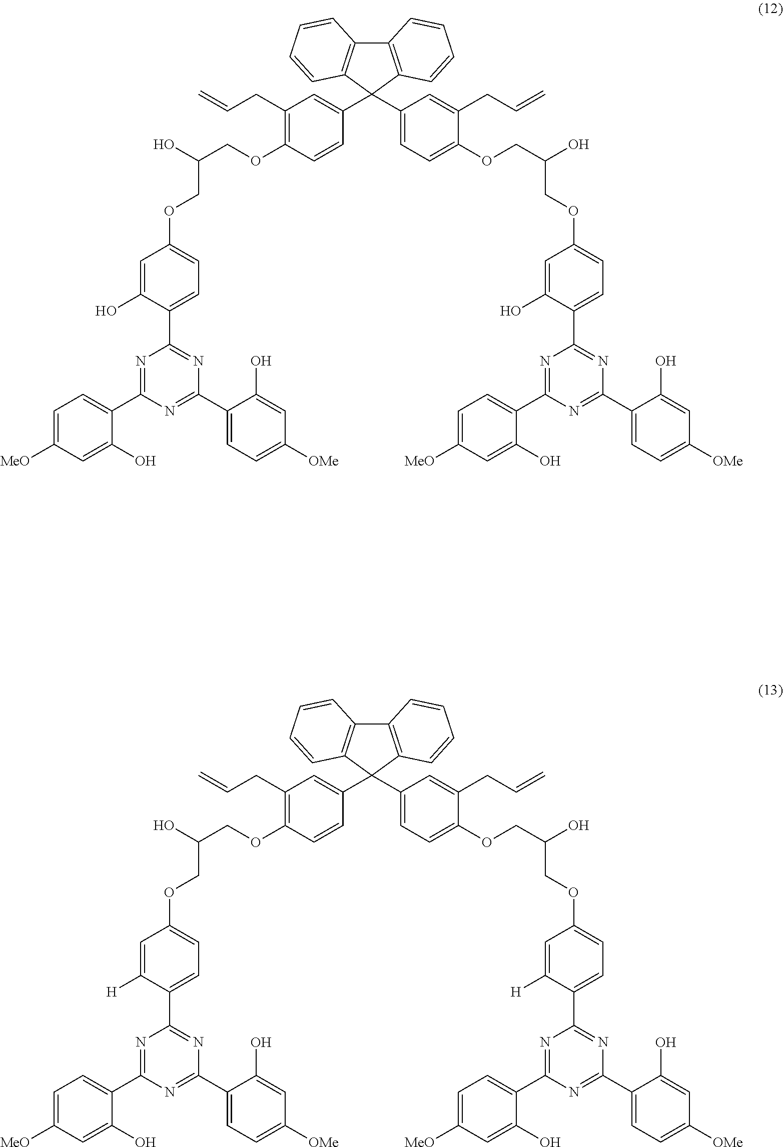

### Method for Manufacturing Inventive Compound

The compound of the general formula (1) can be obtained by reacting a following diallyl-diglycidyl ether compound (14)

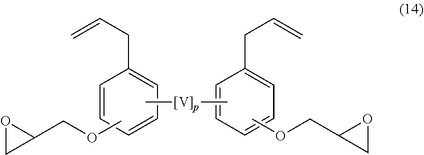

(in the formula (14), V is a divalent organic group selected from any of the following formulae

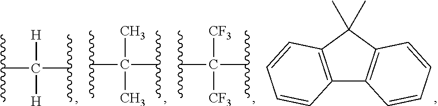

and “p” is 0 or 1.) and 2 equivalents of a compound represented by the following general formula (15)

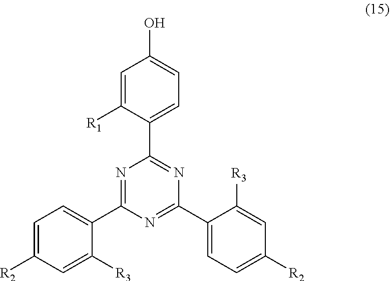

(R¹, R², and R³ are each a hydrogen atom, a hydroxy group, an oxyalkyl group, or an alkyl group, and at least one of R¹ and R³ is a hydroxy group.).

The compound represented by the general formula (1) can be obtained by adding a base to 2 equivalents of the compound represented by the general formula (15) dissolved in a solvent and stirring them; adding 1 equivalent of the diallyl-diglycidyl ether compound (14) to the mixture; and heating and stirring the mixture.

The above solvent is not particularly limited, but aprotic organic solvents are preferable. Examples thereof include ethers such as diethyl ether, tetrahydrofuran, and dioxane; aromatic hydrocarbons such as benzene, toluene, and xylene; halogenated carbonates such as dichloroethane, chloroform, and chlorobenzene; amides such as N,N-dimethylformamide, N,N-dimethylacetamide, N,N-dimethylpropionamide, N-methylpyrrolidone, and N-ethylpyrrolidone; and sulfoxides such as dimethylsulfoxide; etc.

The amount of the solvent is not particularly limited, but is preferably an amount in which the solvent can sufficiently dissolve the compound represented by the general formula (1). It is preferably 0.30M or less, more preferably 0.25M or less, more preferably 0.20M or less, with respect to the compound represented by the general formula (1).

The above-mentioned base is not particularly limited, but it is preferable that the acid dissociation constant (pKa) is larger than that of phenols. Examples thereof include aliphatic amines such as triethylamine, diazabicyclononene, and diazabicycloundecene; heterocyclic amines such as pyridine and N,N-dimethylaminopyridine; ammonium salts such as tetramethylammonium hydroxide, tetraethylammonium hydroxide, tetrapropylammonium hydroxide, and tetrabutylammonium hydroxide; carbonates such as lithium carbonate, sodium carbonate, potassium carbonate, and cesium carbonate; and inorganic salts such as lithium hydroxide, sodium hydroxide, and potassium hydroxide; etc.

The equivalent of the base is not particularly limited, but is preferably 1 equivalent or more, more preferably 1.5 equivalents or more, and most preferably 2 equivalent or more.

The heating temperature is not particularly limited, but is preferably from 25° C. to 180° C., and more preferably from 100° C. to 160° C. The reaction time is about 2 to 30 hours, and preferably 5 to 7 hours.

After completely removing the reaction solvent from these reaction products by reduced pressure-distilling off, an organic solvent and an acidic aqueous solution are added to the reaction system to make the pH of the reaction system less than 7. Next, the acidic aqueous layer is removed; a washing step with pure water is performed; and the solvent used is sufficiently removed by reduced pressure-distilling off to obtain the target compound represented by the general formula (1).

The organic solvents to be added are not particularly limited, but examples thereof include aromatic hydrocarbons such as benzene, toluene, and xylene; aliphatic hydrocarbons such as normal pentane, normal hexane, cyclopentane, and cyclohexane; and halogenated hydrocarbons such as dichloroethane, chloroform, and chlorobenzene; etc. In particular, it is preferable to use toluene.

The acidic aqueous solution used to make the pH of the reaction system less than 7 is not particularly limited, and examples include dilute sulfuric acid, dilute nitric acid, hydrochloric acid, acetic acid, and ammonium chloride aqueous solution, but it is preferable to use divalent dilute sulfuric acid.

### EXAMPLE

Hereinafter, the present invention will be specifically described with reference to Examples and Comparative Examples. However, the present invention is not limited thereto.

### EXAMPLES

238.5 g (600 mmol) of a phenol compound represented by the following formula (16)

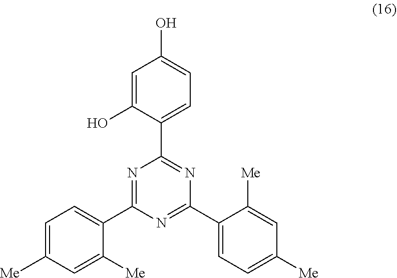 [SMILES: Cc1ccc(-c2nc(-c3ccc(C)cc3C)nc(-c3ccc(O)cc3O)n2)c(C)c1]

(in the general formula (1), R¹═OH, R²═Me, R³═Me) was placed in a 5-liter separable flask equipped with a thermometer and a Dimroth type condenser; 3000 mL of N,N-dimethylformamide as a solvent and 170.0 g (1230 mmol) of potassium carbonate as a base were added; and the mixture was stirred at room temperature for 30 minutes. Thereafter, the mixture was added with 126.2 g (300 mmol) of 2,2′-diallylbisphenol A compound (17)

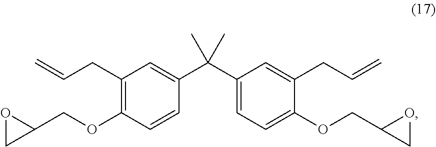 [SMILES: C=CCc1cc(C(C)(C)c2ccc(OCC3CO3)c(CC=C)c2)ccc1OCC1CO1]

and heated at 140° C. to be reacted for 6 hours. After the reaction was completed, N,N-dimethylformamide was sufficiently removed by a reduced pressure-distilling off at 100° C., and 1500 mL of toluene and 2000 mL of pure water were added and stirred. While cooling the reaction solution with ice, 3M dilute sulfuric acid was added dropwise until the pH of the solution became less than 7. The aqueous layer was removed; the solution was washed with pure water until the pH reached about 7; and toluene was completely removed by a reduced pressure-distilling off to obtain 360.9 g (297 mmol) of the target compound (2). The target compound (2) was a reddish-brown viscous substance.

1H nuclear magnetic resonance spectroscopy analysis was performed and it was confirmed that the obtained compound was a target compound (2) in which 2 equivalents of the phenol compound (16) reacted with the 2,2′-diallylbisphenol A compound (17) and an epoxy ring was opened. The results of 1H-NMR are shown in Table 1.

TABLE 1

| Hydrogen | δ, ppm | Integration ratio |
|---|---|---|
| a | 1.35 to 1.69 | 6H |
| k | 2.10 to 2.46 | 12H |
| j | 2.51 to 2.68 | 12H |
| b | 3.19 to 3.29 | 4H |
| e, f, h | 3.85 to 3.25 | 10H |
| d | 4.75 to 5.00 | 4H |
| g | 5.81 to 5.47 | 2H |
| c | 5.72 to 6.94 | 2H |
| E, F | 6.40 to 6.68 | 4H |
| G, I | 6.68 to 6.99 | 8H |
| A, B, C | 6.99 to 7.25 | 6H |
| H | 7.89 to 8.18 | 4H |
| D | 8.30 to 8.42 | 2H |
| i | 10.40 | 2H |

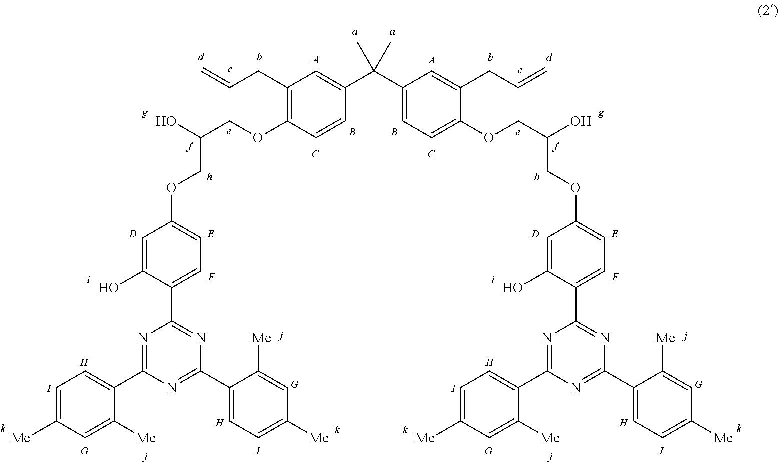

In addition, the absorbance of the obtained compound (2) was measured using an ultraviolet-visible absorption photometer (UV-3600iPlus, manufactured by SHIMADZU Co., Ltd.) in the optical wavelength range of 200 nm to 1000 nm. The maximum absorption wavelengths λmax were 288 nm and 338 nm.

As described above, by using the inventive compound, it was found to be possible to provide a compound having two polymerizable functional groups and two light-absorbing triazine derivatives, which can be used for polymer synthesis. By incorporating the above compound into a polymer of the temporary adhesive material for semiconductors, it can be expected that a light-absorbing component does not elute even when exposed to an organic solvent and maintains the releasing function, and the semiconductor substrate laminated with the temporary adhesive material can be expected to be easily peeled off by light irradiation at the final stage in semiconductor processing.

It should be noted that the present invention is not limited to the above-described embodiments. The embodiments are just examples, and any examples that have substantially the same feature and demonstrate the same functions and effects as those in the technical concept disclosed in claims of the present invention are included in the technical scope of the present invention.

1. A compound represented by the following general formula (1) 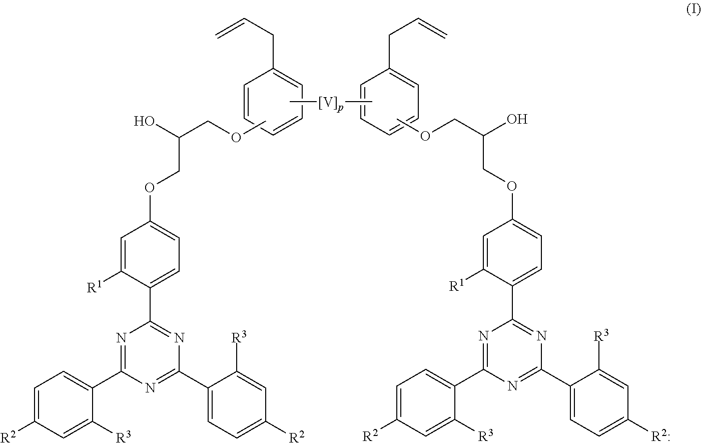
wherein, V is a divalent organic group selected from any of the following general formulae 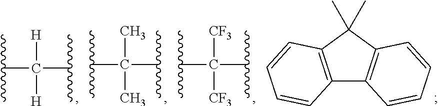
“p” is 0 or 1; R¹, R², and R³ are each a hydrogen atom, a hydroxy group, an oxyalkyl group, or an alkyl group; and at least one of R¹ and R³ is a hydroxy group.

2. The compound according to claim 1, wherein the compound is a light-absorbing compound.

3. The compound according to claim 2, wherein the compound has one or more maximum absorption wavelengths λmax of light between 200 nm and 1000 nm.
